(19) United States
(12) Patent Application Publication
(10) Pub. No.: US 2009/0253990 A1
(43) Pub. Date: Oct. 8, 2009

(54) OPTICAL DIAGNOSIS OF HEMOPHILIC JOINT EFFUSION

(75) Inventors: Chad Allen Lieber, Chino Hills, CA (US); Diane Jean Nugent, San Juan Capistrano, CA (US); Amit Soni, Irvine, CA (US)

Correspondence Address: KNOBBE MARTENS OLSON & BEAR LLP, 2040 MAIN STREET, FOURTEENTH FLOOR, IRVINE, CA, 92614, US

(73) Assignee: Children's Hospital of Orange County, Orange, CA (US)

(21) Appl. No.: 12/329,311

(22) Filed: Dec. 5, 2008

Related U.S. Application Data
(60) Provisional application No. 61/012,004, filed on Dec. 6, 2007.

(51) Int. Cl. A61B 6/00

(52) U.S. Cl. 600/476

(57) ABSTRACT

Non-invasive systems and methods to distinguish between blood and synovial fluid in patients are described. In one embodiment, the system comprises a patient interface system that can be placed over a swollen joint. A region of the swollen joint is illuminated with radiation. The scattered or transmitted radiation from the region of effusion is collected by a collection system and detected by a radiation detector. The information from the detector is analyzed by an analytic processing system to diagnose the cause of the joint effusion.

### CROSS-REFERENCE TO RELATED APPLICATIONS

This application claims priority to U.S. Provisional Application No. 61/012,004 filed on Dec. 6, 2007 titled “OPTICAL DIAGNOSIS OF HEMOPHILIC JOINT EFFUSION” (Atty. Docket No. CHIHO.032PR) which is hereby expressly incorporated by reference in its entirety.

### BACKGROUND

1. Field of the Invention

This invention relates in general to optical diagnostic systems and methods. In particular some aspects of this invention relate to the use of optical diagnostic systems and methods to determine the cause of joint inflammation.

2. Description of the Related Art

In patients with bleeding disorders, effusion of blood in the joints can be a common experience. Such joint bleeds can occur spontaneously or can be caused by trauma or other joint related conditions. These bleeds can eventually cause joint damage as enzymes in the blood erode the joint and bone growth is altered in a vicious cycle. The joint bleeds can result in joint inflammation which can require treatment.

However, the cause of joint inflammation is not always blood. For example, arthritic patients can also often experience effusion of synovial fluid in the joints leading to joint inflammation. While clinical management of a bleeding joint necessitates treatment with specialized drugs to curtail and remove the blood from the joint space, synovial effusion is better managed by the body and clinical treatment generally consists of inexpensive pain medication.

The cost difference between the treatments is substantial. Thus in arthritic patients with bleeding disorders, it is cost effective to identify whether the joint inflammation is caused by blood or by synovial fluid. For these patients, the general method of identifying the cause of joint inflammation is magnetic resonance imaging (MRI). However, MRI is generally more time consuming and expensive than providing treatment for bleeding joints. Thus there is a need for a fast and inexpensive technique to determine whether the joint inflammation is caused by blood or synovial fluid.

Optical techniques have a growing track record of successful application in noninvasive medical diagnostics. In general, such techniques use light of specific wavelengths or wavelength regions to illuminate a sample of interest, such that the material properties of the illuminated sample can be deduced via the light that is absorbed, reflected or altered by the sample and measured with optical detectors. A variety of optical techniques have been used for medical applications such as diffuse reflectance, transmission spectroscopy, fluorescence spectroscopy and Raman spectroscopy. Very few attempts to utilize optical techniques for characterizing effusions have been made.

### SUMMARY

Various embodiments described herein comprise systems and methods to determine the source of joint inflammation using optical diagnostics. In one embodiment, a system to provide optical diagnosis of the source of joint inflammation is disclosed. The system comprises an illumination system; a patient interface configured to be placed at a distance from a patient's joint; a collection system; and an analytic processing system, wherein the system is configured to automatically distinguish between blood and synovial fluid.

In one embodiment, a method to determine the source of joint inflammation in a patient is disclosed, the method comprises impinging electromagnetic radiation on a portion of the inflamed surface of the joint; collecting electromagnetic radiation scattered from or transmitted through the inflamed surface of the joint; detecting said electromagnetic radiation scattered or transmitted from the inflamed surface of the joint using a radiation detector; recording a frequency spectrum of the detected electromagnetic radiation; comparing the recorded frequency spectrum to a collection of known frequency spectra from a plurality of known sources; and identifying the source of joint inflammation as the known source whose frequency spectrum most closely matches the recorded frequency spectrum.

### BRIEF DESCRIPTION OF THE DRAWINGS

FIG. 1 illustrates a system for optical diagnosis of source of joint inflammation.

FIG. 2 indicates a preferred method to perform optical diagnosis of hemophilic joint effusion

FIG. 3 illustrates a system for optical diagnosis of source of joint inflammation including optical waveguides.

FIG. 4 illustrates a compact system for optical diagnosis of source of joint inflammation.

### DETAILED DESCRIPTION OF THE FIGURES

Although certain preferred embodiments and examples are disclosed below, inventive subject matter extends beyond the specifically disclosed embodiments to other alternative embodiments and/or uses of the invention, and to modifications and equivalents thereof. Thus, the scope of the inventions disclosed herein is not limited by any of the particular embodiments described below. For example, in any method or process disclosed herein, the acts or operations of the method or process may be performed in any suitable sequence and are not necessarily limited to any particular disclosed sequence. For purposes of contrasting various embodiments with the prior art, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. The systems and methods discussed herein can be used anywhere, including, for example, in laboratories, hospitals, healthcare facilities, intensive care units (ICUs), or residences. Moreover, the systems and methods discussed herein can be used for invasive techniques, as well as non-invasive techniques or techniques that do not involve a body or a patient.

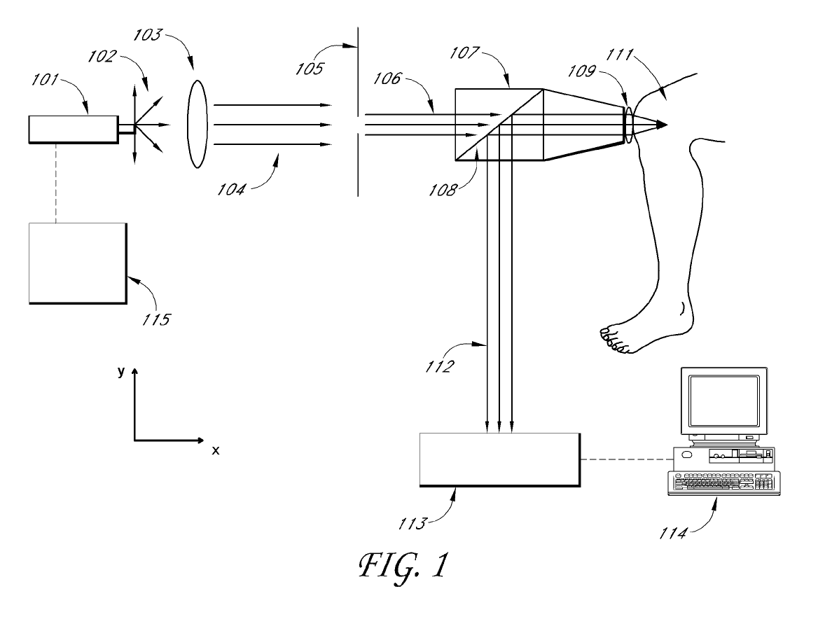

FIG. 1 illustrates a system for optical diagnosis of the source of joint inflammation. The system is configured to emit electromagnetic radiation in a certain wavelength range. The electromagnetic radiation can interact with a source of analyte 111. In some embodiments, the source of analyte 111 can be an inflamed joint in a patient. In some other embodiments, the source of analyte 111 can be a sample of biological fluid. In some embodiments, the analyte interacting with the electromagnetic radiation can be blood, synovial fluid, or both.

The system illustrated in FIG. 1 can be used in hospitals, urgent care centers, emergency rooms, homes, laboratories, etc. The system can be mobile and easily portable. The system can be used and operated by nurses, doctors, residents and the patients. In some embodiments, the system can be automated and designed in such a manner that it can be operated by an approximately untrained operator. In some embodiments, the system can be setup and operated in a relatively short duration.

The system illustrated in FIG. 1 comprises an illumination system, a patient interface system 107, a collection system and an analytic processing system 114. The illumination system can comprise a source of electromagnetic radiation 101. The source of electromagnetic radiation 101 can emit light, heat or both. In some embodiments, the source of electromagnetic radiation 101 can emit other types of radiation such as high energy particles. The source of electromagnetic radiation 101 can include an incandescent lamp, light emitting diode (LED), laser diode, lasers, etc. The source of electromagnetic radiation 101 can emit radiation in a wavelength range between 400-2000 nanometer. In some embodiments the electromagnetic radiation 101 can be less than 400 nanometer or greater than 2000 nanometer. In some embodiments, the source of electromagnetic radiation 101 can emit radiation in broadband spectral range, in continuous spectral range or in discrete bands in the wavelength region between 600-1400 nanometer.

The source of electromagnetic radiation 101 can be operated in continuous mode or in pulsed mode. In some embodiments, electric power to the source of electromagnetic radiation 101 can be supplied from an electrical power supply line. In various embodiments, electrical power to the source of electromagnetic radiation 101 can be supplied by a voltage regulator. In some embodiments, electrical power to the source of electromagnetic radiation 101 can be supplied by a battery pack. The source of electromagnetic radiation can be controlled by an external controller 115 as shown in FIG. 1. The external controller 115 can switch the source of electromagnetic radiation 101 on or off. In some embodiments, the external controller 115 can be used to alternate between continuous and pulsed mode of operation. The external controller 115 can also be used to change the wavelength and/or the power of the electromagnetic radiation emitted by the source 101.

The electromagnetic radiation emitted by the source 101 can be emitted in all directions as illustrated by the group of light rays 102. In some embodiments however the electromagnetic radiation can be directional. In some embodiments, the electromagnetic radiation can be directed substantially parallel to the optical axis of the system, for example parallel to +x direction in FIG. 1. In some other embodiments, the electromagnetic radiation emitted from the source can be coherent and directional. The electromagnetic radiation emitted from the source 101 can be focused by a lens system 103. The lens system 103 can comprise a single lens or multiple lenses. Additionally, the lens system 103 can include electromagnetic radiation filters, beam splitters, mirrors, polarizers, prisms and other optical components. In some embodiments, the focused beam 104 can be spatially filtered by a slit or pinhole 105. The electromagnetic radiation 106 that is shaped and conditioned in the above described manner can be directed into a patient interface system 107.

In various embodiments, the patient interface system 107 can be flexible or rigid. In some embodiments, the patient interface system 107 can comprise thermoplastic material. In some embodiments, the patient interface system can comprise rubber, silicone, aluminum, stainless steel or other such materials. The patient interface system 107 can be placed at a distance from the source of analyte 111 or can be attached to the source of analyte 111. For example, as shown in FIG. 1 the patient interface system 107 can be placed at or near the knee joint of a patient. In various embodiments, the patient interface system 107 can be placed at or near any other joint of the human body such as the elbow joint. The patient interface system 107 can be held in place by a patient interface holder, not shown. In some embodiments, the patient interface system 107 can be attached to the source of analyte 111 by pressure, adhesive or suction. In some embodiments, the patient interface system 107 can be disposed on the source of analyte 111. In various embodiments, the patient interface system can be placed at a distance ranging from approximately 0 cm to approximately 30 cm from the source of analyte 111. In some embodiments, the patient interface system may be placed at a distance of approximately 0 cm to approximately 10 cm from the source of analyte 111.

In various embodiments, the patient interface system 107 can comprise optical components such as lens systems, reflecting optics, beam splitters, mirrors, prisms, etc. In some embodiments, the beam of electromagnetic radiation 106 from the source 101 can be directed towards the source of analyte 111 (for example, the knee joint of a patient) by a partially reflecting mirror 108 which transmits radiation propagating parallel to the +x direction. The electromagnetic radiation can be focused on a portion of the inflamed joint in a patient by another lens system 109. The position of the patient interface system 107 can be adjusted either manually or automatically to accommodate for different joints and skin depth. In some embodiments, the patient interface system 107 can make noninvasive measurements and can be painless when used in connection with a patient. In some embodiments, however the patient interface system can be inserted into the body of a patient through the skin, for example, using a catheter. The optical diagnostic system further comprises a detection system 113 and an analytic processing system 114. The detection system 113 can comprise photodiodes, charge-coupled device (CCD), photodiode arrays, complementary metal oxide semiconductor (CMOS) detectors, photomultiplier tubes, etc. The analytic processing system 114 can comprise a microprocessor. The processing in the analytical processing system 114 can occur via software written for use on a computer, microcomputer or by algorithms written directly into processing chips (such as erasable programmable read only memory (EPROM)).

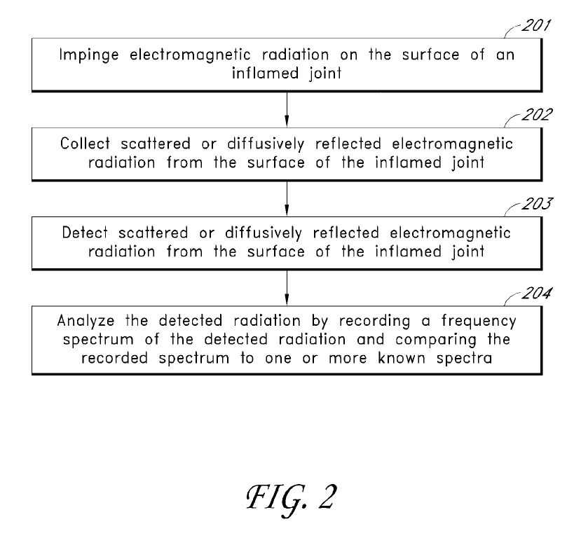

FIG. 2 describes a method of determining the source of joint inflammation. The method comprises the following steps as described below. In step 201, electromagnetic radiation is impinged on a portion of the inflamed joint from the patient interface system 107. The electromagnetic radiation can interact with the analyte (for example blood or synovial fluid) in the inflamed joint of the patient. The characteristics of the electromagnetic radiation may be altered by the analyte. For example interaction with the analyte can change the frequency spectrum or the intensity of the electromagnetic radiation. Upon interaction, the electromagnetic radiation can be scattered by or transmitted through the surface of the inflamed joint. The scattered or transmitted electromagnetic radiation after interaction with the analyte is collected by the patient interface system 107 as shown in step 202. Collection optics such as high numerical aperture lenses, prisms, etc can be used to collect the scattered or transmitted radiation from the surface of the inflamed joint. In some embodiments, as shown in FIG. 1, the scattered or transmitted electromagnetic radiation from the inflamed joint 111 is directed along the same path as the incident radiation. At the surface of the beam splitter or partially reflecting mirror 108, the electromagnetic radiation after interaction with the analyte is reflected by the partially reflecting mirror 108 and directed parallel to the −y direction towards the detection system 113 as shown by the light path 112. In step 203, the collected scattered or transmitted radiation is detected with the detection system 113. In some embodiments, the detection system 113 along with the components used to direct the beam of electromagnetic radiation towards the detection system can be a part of a collection system. In some embodiments, the detection system 113 can measure and record the spectrum, intensity or other characteristics of the electromagnetic radiation after interaction with the analyte as shown in step 204. In some embodiments, a reference signal can be provided to the detection system 113 to provide a baseline to identify the changes in the characteristics of the electromagnetic radiation after interaction with the analyte.

As shown in step 204, the information from the detection system 113 is transported to an analytic processing system 114. The analytic processing system 114 can perform qualitative and/or quantitative assessment of the information from the detection system 113. Statistical procedures can be employed that compare the electromagnetic radiation detected at specific frequencies and correlate this information with absorption peaks in known analytes such as blood or synovial fluid. For example, in some embodiments, the spectrum of the scattered or transmitted radiation from the surface of an inflamed joint can be compared to one or more known spectra (e.g., spectra of blood and synovial fluid) by taking a ratio. In some other embodiments, a cross correlation function can be calculated between the spectrum of the scattered or diffusely reflected radiation and one or more known spectra (e.g., spectra of blood and synovial fluid). Other methods such as linear or non-linear combinations of the spectrum of the scattered or diffusely reflected radiation can be used to determine the nature of the analyte. Other statistical methods such as regression analysis can also be used to obtain relevant information from the scattered or diffusely reflected radiation. In some embodiments, the systems and methods outlined above can be used to differentiate relative quantities of oxygenated and deoxygenated hemoglobin to ascertain the source or age of the blood.

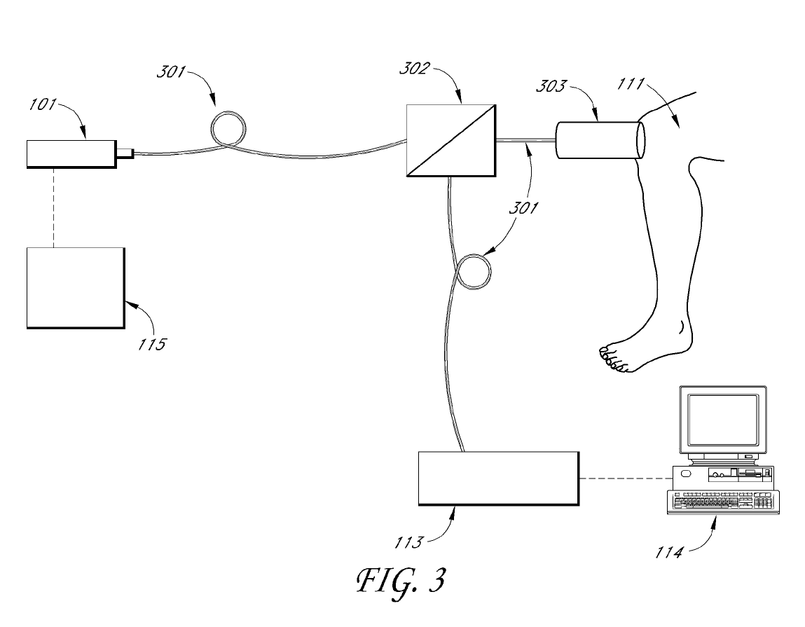

In some embodiments, the system of FIG. 1, can comprise electromagnetic waveguides such as optical fibers, hollow waveguides, silica waveguides or liquid waveguides to transport electromagnetic radiation in part from the source 101 to the source of analyte 111 and from the source of the analyte 111 to the collection system. For example, as illustrated in FIG. 3, an optical fiber 301 can be used to deliver light from the source 101 to a beam splitter 302. The beam splitter 302 can be provided with optical fibers 301 as well. The patient interface system 303 can comprise optical fibers, microlens, collimators, retroreflectors, etc. The detection system 113 can be provided with optical fibers or optical fiber terminations. The use of optical fibers and other waveguides can be advantageous in making the system compact, robust, mobile, automated, etc.

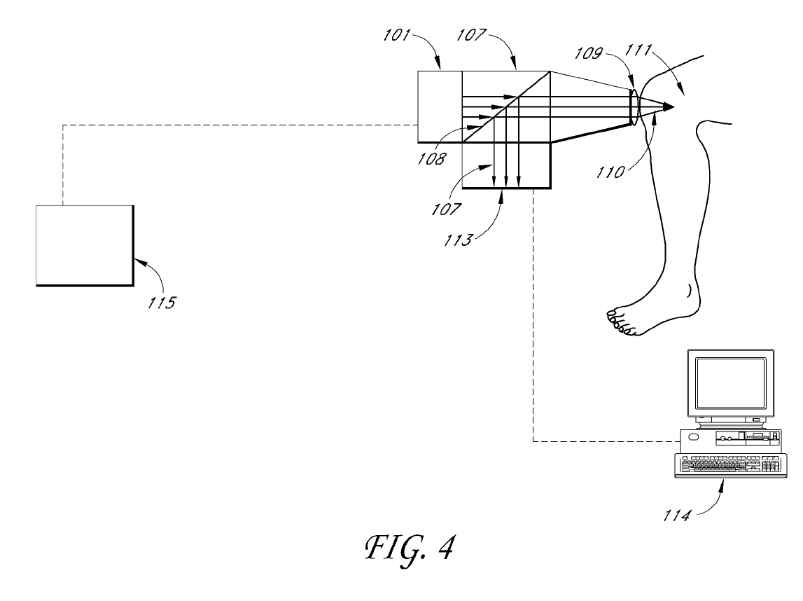

In some embodiments, as shown in FIG. 4, the source of electromagnetic radiation 101 and/or the detection system 113 can be integrated with the patient interface system 107. The optical elements used to shape and process the electromagnetic radiation can be embedded in the patient interface system 107. The integrated system can be provided with electrical wires and cables to supply power and connect the analytic processing system.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the Claims that follow.

What is claimed is:

1. A system to provide optical diagnosis of the source of joint inflammation, the system comprising:
an illumination system;
a patient interface system configured to be placed at a distance from a joint in a patient;
a collection system; and
an analytic processing system,
wherein the system is configured to noninvasively distinguish between blood and synovial fluid.

2. The system of claim 1, wherein the illumination system comprises:
a source of electromagnetic radiation;
a radiation delivery system configured to process the electromagnetic radiation from the source;

3. The system of claim 2, wherein the source of electromagnetic is configured to emit radiation having wavelengths between 600 nanometers and 1400 nanometers.

4. The system of claim 2, wherein the source of electromagnetic radiation is configured to be operated in pulsed mode.

5. The system of claim 2, wherein the radiation delivery system comprises lens.

6. The system of claim 2, wherein the radiation delivery system comprises mirrors.

7. The system of claim 2, wherein the radiation delivery system comprises filters.

8. The system of claim 2, wherein the radiation delivery system comprises waveguides.

9. The system of claim 1, wherein the distance between the patient interface system and the joint in a patient can vary between approximately 0 cm and 10 cm.

10. The system of claim 1, wherein the patient interface system is disposed on the joint in a patient.

11. The system of claim 1, wherein the patient interface system comprises adhesive tape.

12. The system of claim 1, wherein the patient interface system comprises a suction element.

13. The system of claim 1, wherein the patient interface system comprises a material selected from a group consisting of thermoplastic, rubber, silicone, aluminum and stainless steel.

14. The system of claim 1, wherein the patient interface system comprises an optical component selected from a group consisting of lens, mirrors, prisms and reflectors.

15. The system of claim 1, wherein the patient interface system comprises optical fibers.

16. The system of claim 1, wherein the collection system comprises a photodetector.

17. The system of claim 1 wherein the collection system further comprises elements to collect radiation from the source of analyte.

18. The system of claim 1, wherein the analytic processing system comprises a microprocessor.

19. A method to determine the source of joint inflammation in a patient, the method comprising:
impinging electromagnetic radiation on a portion of an inflamed surface of the joint;
collecting electromagnetic radiation scattered by or transmitted through the inflamed surface of the joint;
detecting said electromagnetic radiation scattered by or transmitted through the inflamed surface of the joint using a radiation detector;
recording a frequency spectrum of the detected electromagnetic radiation; and
comparing the recorded frequency spectrum to a collection of known frequency spectra from a plurality of known sources; and
identifying the source of joint inflammation as the known source whose frequency spectrum most closely matches the recorded frequency spectrum.

20. The method of claim 19, wherein comparing the recorded frequency spectrum to a collection of known frequency spectra comprises taking a ratio of the recorded frequency spectrum to one or more of the known frequency spectra at one or more specific frequencies.

21. The method of claim 19, wherein comparing the recorded frequency spectrum to a collection of known frequency spectra comprises calculating a cross correlation function between the recorded frequency spectrum and one or more of the known frequency spectra.

22. The method of claim 19, wherein the collection of known frequency spectra comprises absorption features of at least one of blood and synovial fluid.

23. The method of claim 19, wherein impinging electromagnetic radiation comprises:
emitting electromagnetic radiation from a source of electromagnetic radiation; and
directing the emitted electromagnetic radiation onto the inflamed surface of the joint through a patient interface system.

24. The method of claim 23, wherein the patient interface system comprises an optical component selected from a group consisting of lens, mirrors, prisms and reflectors.

25. The method of claim 19, wherein electromagnetic radiation scattered by or transmitted through the inflamed surface of the joint is collected using a collection system comprising optical elements.

26. The method of claim 19, wherein the radiation detector comprises a photodetector.
